(19) United States
(12) Patent Application Publication
(10) Pub. No.: US 2026/0060698 A1
(43) Pub. Date: Mar. 5, 2026

(54) CONTRAST INJECTION AND VISUALIZATION SYSTEMS AND METHODS FOR THROMBUS REMOVAL DEVICE

(71) Applicant: Shifamed Holdings, LLC, Campbell, CA (US)

(72) Inventors: Aadel AL-JADDA, San Carlos, CA (US); Uday ILLINDALA, Los Gatos, CA (US); Paul GUNNING, Montara, CA (US); Kevin MULLER, San Carlos, CA (US); Amr SALAHIEH, Saratoga, CA (US); Praveen Krishna DALA, San Ramon, CA (US)

(21) Appl. No.: 19/106,174

(22) PCT Filed: Aug. 24, 2023
(86) PCT No.: PCT/US2023/072853

§ 371 Date: Feb. 24, 2025

Related U.S. Application Data
(60) Provisional application No. 63/373,430, filed on Aug. 24, 2022.
(60) Provisional application No. 63/380,984, filed on Oct. 26, 2022.

(51) Int. Cl. A61B 17/22; A61B 6/00; A61B 17/00; A61B 34/20

(52) U.S. Cl. CPC A61B 17/22; A61B 6/481; A61B 34/20; A61B 2017/00778; A61B 2017/22087

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application No. 63/373,430, titled “THROMBUS REMOVAL SYSTEMS AND ASSOCIATED METHODS”, filed on Aug. 24, 2022, and U.S. provisional patent application No. 63/380,984, titled “CONTRAST INJECTION AND VISUALIZATION SYSTEMS AND METHODS FOR THROMBUS REMOVAL DEVICE”, filed on Oct. 26, 2022, which are both herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs.

Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure.

Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days.

Visualization of clots and the location of a thrombectomy device relative to the clot is also an issue. While positioning a thrombectomy catheter in the vicinity of a clot is fairly routine, is particularly difficult to properly position a thrombectomy catheter at the clot locally. The further away the catheter is from the clot prior to turning on aspiration, the more blood is likely to be unnecessarily aspirated.

More recently other tools like clot retrievers have been developed to treat DVT and pulmonary embolism (PE), but these tools are not being widely adopted because of their limited effectiveness and additional costs versus aspiration or the standard of case.

Other recent developments focus on slicing or macerating the clot, but these mechanisms are designed to reduce the risk of the catheter clogging and do not address the problem of tough, large, organized clots.

There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4H illustrate various embodiments of a thrombus removal system including a saline source, an aspiration system, and one or more controls for controlling irrigation and/or aspiration of the system.

FIG. 5A-5E illustrate one embodiment of a thrombus removal device.

FIGS. 6A-6J illustrate a sequence of advancing a delivery catheter and dilator to a target thrombus location within a patient.

FIG. 7 an example of an introducer catheter and a dilator with grooves configured to deliver contrast into a target location such as the pulmonary artery.

FIG. 8 is a flowchart illustrating a method of visualizing a thrombectomy procedure.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A method of visualizing a thrombectomy procedure is provided, comprising: advancing a thrombus removal device to a target thrombus location; injecting a volume of contrast near the target thrombus location through one or more fluid lumens and fluid ports of the thrombus removal device; visualizing the volume of contrast to identify a thrombus; capturing the thrombus in a funnel of the thrombus removal device; and delivering a jetting fluid from a fluid source into the one or more fluid lumens to produce one or more jet streams from the one or more fluid ports to cut the thrombus.

In one aspect, injecting the volume of contrast further comprises injecting the volume of contrast with a contrast injector of the thrombus removal device.

In one aspect, the volume of contrast enters the one or more fluid lumens distal to the fluid source.

In one aspect, the volume of contrast enters the one or more fluid lumens proximal to the fluid source.

In some aspects, the volume of contrast enters the one or more fluid lumens at the fluid source.

In one aspect, the volume of contrast comprises less than 5 ml.

In one aspect, the volume of contrast comprises less than 10 ml.

In one aspect, the volume of contrast comprises less than 20 ml.

In some aspects, the method includes repeating the injecting and visualizing steps periodically.

In one aspect, the method includes repeating the injecting and visualizing steps every 1-3 seconds.

In other aspects, the method includes repeating the injecting and visualizing steps every 3-5 seconds.

In one aspect, the method includes adjusting a concentration of the contrast with the jetting fluid.

In some aspects, the method includes delivering two or more concentration densities of contrast into the target thrombus location to produce a gradient of images of the thrombus.

In another aspect, the method includes re-positioning the thrombus removal device adjacent to the thrombus based on the visualizing.

In one aspect, the target thrombus location is located within a pulmonary artery.

A method of visualizing a thrombectomy procedure, comprising: advancing a thrombus removal device to a target thrombus location; injecting a volume of contrast near the target thrombus location through one or more contrast lumens and contrast ports of the thrombus removal device; visualizing the volume of contrast to identify a thrombus; capturing the thrombus in a funnel of the thrombus removal device; and delivering a jetting fluid from a fluid source into one or more fluid lumens to produce one or more jet streams from one or more fluid ports to cut the thrombus.

In some aspects, injecting the volume of contrast further comprises injecting the bolus of contrast with a contrast injector of the thrombus removal device.

In one aspect, the volume of contrast comprises less than 5 ml.

In one aspect, the volume of contrast comprises less than 10 ml.

In another aspect, the volume of contrast comprises less than 20 ml.

In some aspects, the method includes repeating the injecting and visualizing steps periodically.

In some aspects, the method includes repeating the injecting and visualizing steps every 1-3 seconds.

In some aspects, the method includes repeating the injecting and visualizing steps every 3-5 seconds.

In one aspect, the one or more contrast lumens and contrast ports are distinct from the one or more fluid lumens and fluid ports.

In some aspects, the method includes delivering two or more concentration densities of contrast into the target thrombus location to produce a gradient of images of the thrombus.

In another aspect, the method includes re-positioning the thrombus removal device adjacent to the thrombus based on the visualizing.

In one aspect, the target thrombus location is located within a pulmonary artery.

A method of visualizing a thrombectomy procedure is provided, comprising: advancing an introducer sheath and a dilator to a target thrombus location; delivering a volume of contrast near the target thrombus location from between the introducer sheath and the dilator; visualizing the volume of contrast to identify a thrombus; removing the dilator from the introducer sheath; introducing a thrombus removal device into the introducer sheath; advancing the thrombus removal past a distal opening of the introducer sheath to deploy a funnel of the thrombus removal device; capturing the thrombus in the funnel; and delivering a jetting fluid from a fluid source into one or more fluid lumens and one or more jet ports of the thrombus removal device to produce one or more jet streams to cut the thrombus.

In one aspect, the method includes aspirating the thrombus with the thrombus removal device.

In one aspect, the target thrombus location is in a pulmonary artery of a subject.

In another aspect, the contrast is delivered from grooves or slits in the dilator.

In one aspect, the method includes delivering a volume of contrast near the target thrombus location from the introducer sheath when the funnel of the thrombus removal device is deployed.

In some aspects, delivering the volume of contrast further comprises injecting the volume of contrast with a contrast injector of the thrombus removal device.

In one aspect, the volume of contrast comprises less than 5 ml.

In one aspect, the volume of contrast comprises less than 10 ml.

In one aspect, the volume of contrast comprises less than 20 ml.

In another aspect, the method includes repeating the delivering and visualizing steps periodically.

In another aspect, the method includes repeating the delivering and visualizing steps every 1-3 seconds.

In another aspect, the method includes repeating the delivering and visualizing steps every 3-5 seconds.

In another aspect, the method includes delivering two or more concentration densities of contrast into the target thrombus location to produce a gradient of images of the thrombus.

In another aspect, the method includes re-positioning the thrombus removal device adjacent to the thrombus based on the visualizing.

A thrombectomy catheter device is provided, comprising: an elongate catheter shaft; a funnel disposed on or near a distal end of the shaft; an aspiration lumen in the shaft; at least one fluid lumen coupled to a fluid source; at least one port disposed near a distal end of the at least one fluid lumen, the at least one port configured to produce a jetted fluid stream to macerate or cut a target thrombus; and a contrast injector fluidly coupled to the shaft, the contrast injector being configured to deliver a bolus of contrast from the shaft into a target thrombus location.

In some aspects, the contrast injector is fluidly coupled to the at least one fluid lumen distal to the fluid source.

In one aspect, the contrast injector is fluidly coupled to the at least one fluid lumen proximal to the fluid source.

In one aspect, the contrast injector is fluidly coupled to the at least one fluid lumen at the fluid source.

In one aspect, the system includes one or more valves selectively controllable to allow contrast from the injector to enter the shaft of the thrombus removal device.

In one aspect, the one or more valves and/or the injector are configured to deliver selected volumes of contrast at selected time intervals into the shaft.

A contrast delivery system is provided, comprising: an elongate, steerable shaft; a lumen disposed within the shaft; a dilator assembly removably disposed within the shaft; and a contrast source in fluid communication with the lumen, the contrast source being configured to deliver a volume of contrast to a target thrombus location between the dilator assembly and the shaft.

In one aspect, the dilator assembly comprises one or more grooves where it interfaces with the shaft.

In one aspect, the dilator assembly comprises one or more slits where it interfaces with the shaft.

In another aspect, the dilator assembly comprises one or more ports configured to deliver the volume of contrast.

In one aspect, the contrast source comprises a contrast injector.

In some aspects, the contrast injector is configured to deliver a bolus of contrast less than 5 ml in volume.

In one aspect, the contrast injector is configured to deliver a bolus of contrast less than 10 ml in volume.

In one aspect, the contrast injector is configured to deliver a bolus of contrast less than 20 ml in volume.

In another aspect, the contrast injector is configured to deliver the volume of contrast periodically.

A medical device loading tool is provided, comprising: a funnel introducer comprising a distal opening and a proximal opening; and an introducer shuttle comprising an elongate shaft adapted to be inserted into the proximal opening; wherein a thrombectomy catheter having an expandable and collapsible funnel is configured to be loaded into the introducer funnel in a delivery configuration by pulling the thrombectomy catheter and funnel proximally through the funnel introducer and into the introducer shuttle.

In one aspect, the funnel introducer is configured to reduce a loading force required to sheath the funnel into the introducer shuttle.

In another aspect, the funnel introducer comprises a chamfered edge inside the distal opening and configured to provide a smooth lead-in from the funnel introducer into the introducer shuttle.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

Alternatively, according to additional embodiments of the present technology, the fluid delivery mechanism can provide a plurality of contrast-containing fluid streams (e.g., contrast jets) to fluid apertures of the thrombus removal system for the introduction of a contrast agent or dye into the patient from the fluid apertures of the thrombus removal system. In some embodiments, the contrast-containing fluid streams or contrast jets can be provided at a sufficient pressure or velocity for macerating, cutting, fragmenting, pulverizing and/or urging thrombus to be removed from a proximal portion of the thrombus removal system. The contrast-containing fluid streams can serve the dual-purpose of breaking the thrombus up into smaller pieces and also providing visualization of the thrombectomy procedure and clot (e.g., in real-time during the procedure). For example, the contrast containing fluid streams can comprise a concentration of radiopaque material.

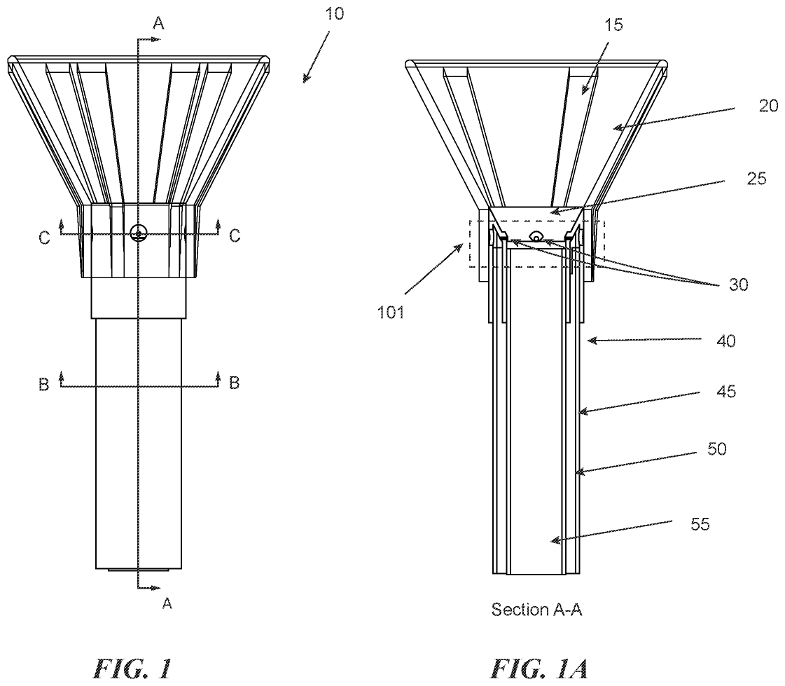

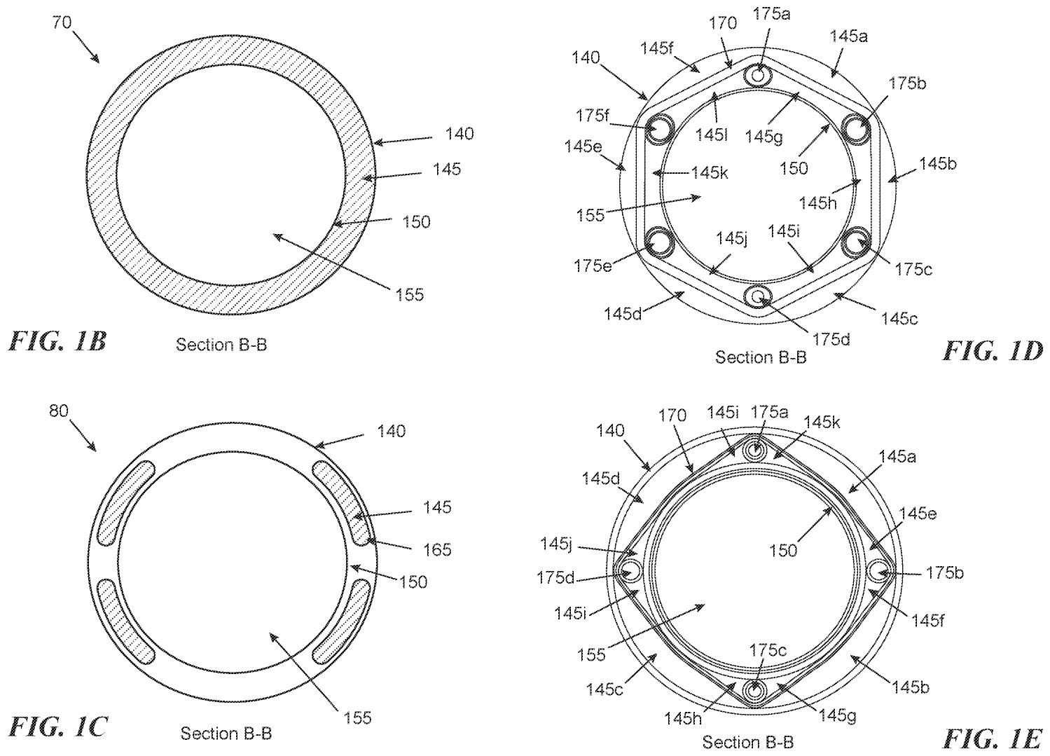

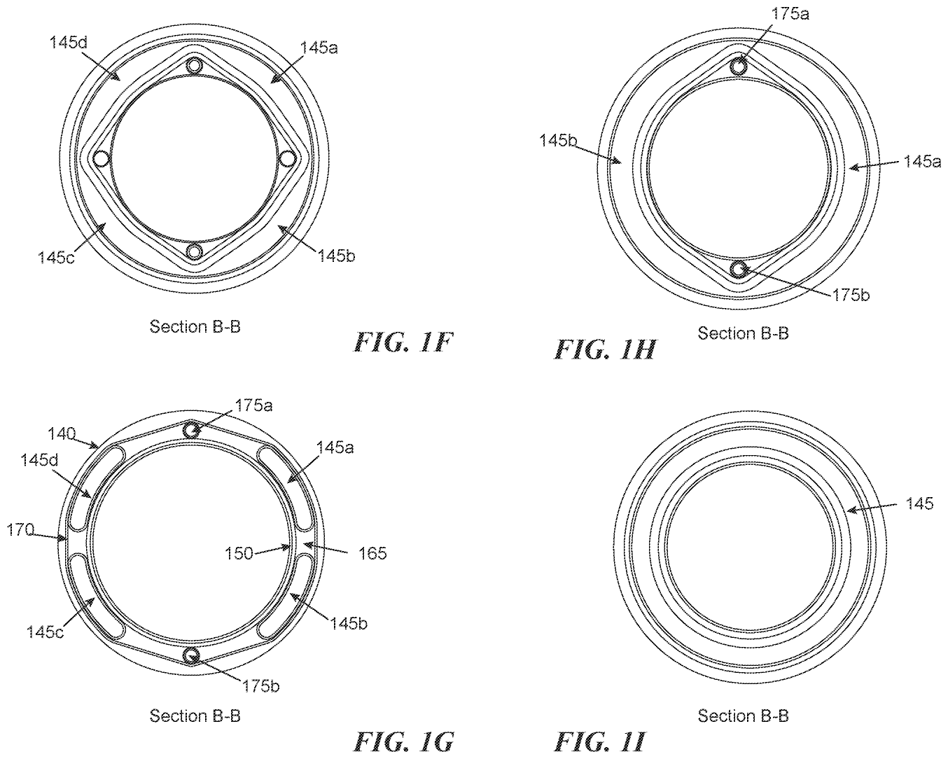

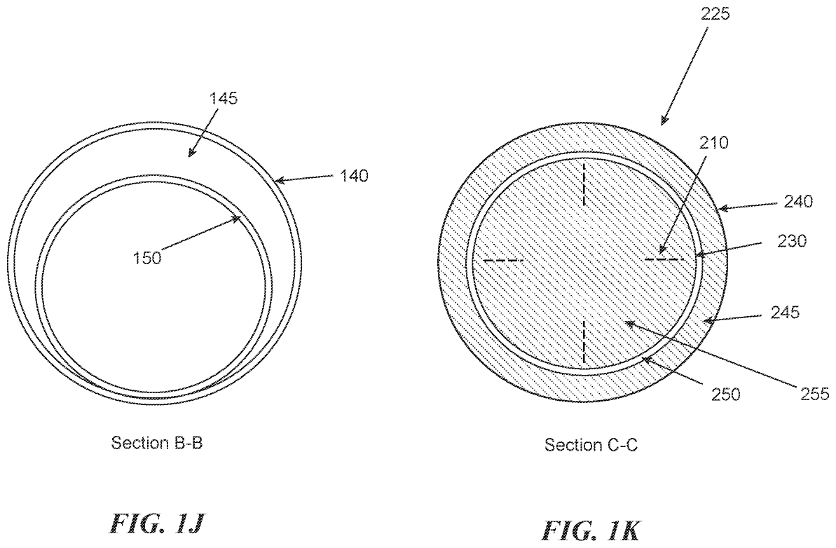

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment IF includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

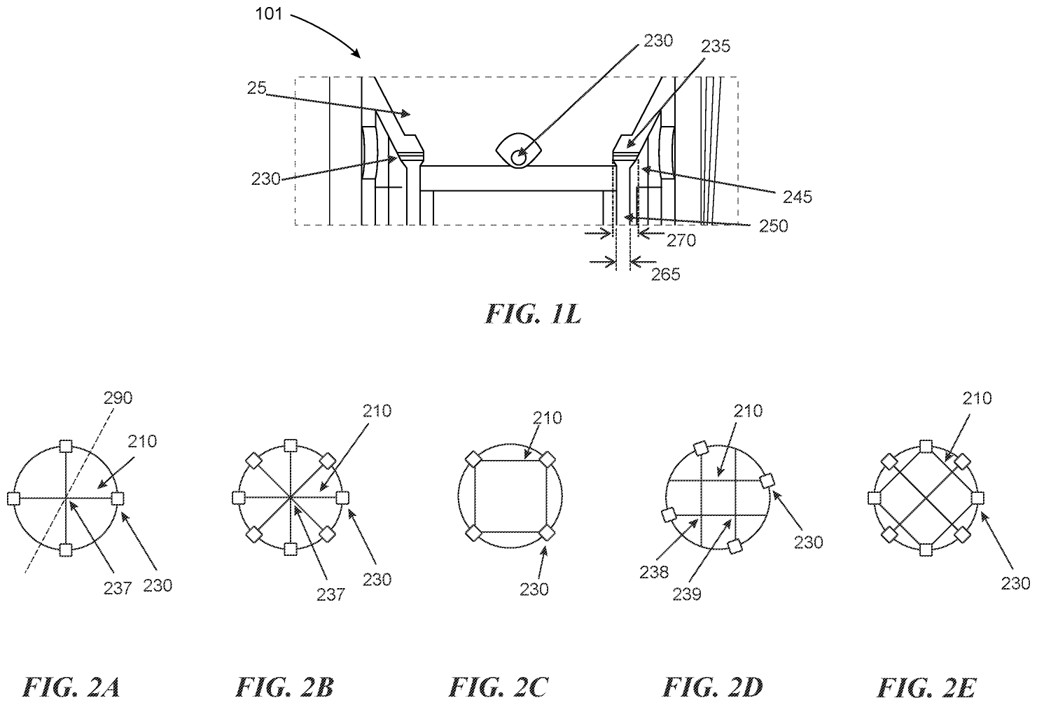

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

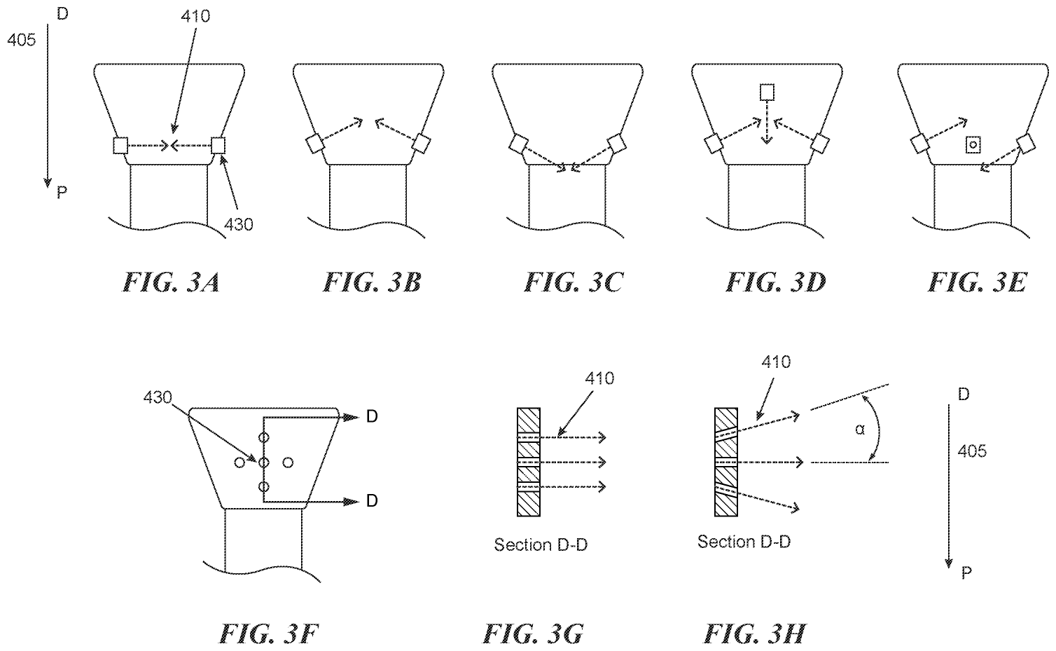

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

### Cavitation Generation

The exemplary system includes fluidic jets configured in a particular manner to enhance removal of clot. The exemplary fluid streams or jets have been shown in bench studies to dramatically improve removal of clot through various mechanisms of action optionally including, but not limited to, cavitation and water cutting. In contrast to conventional fluid mechanisms for thrombectomy, in some embodiments herein, fluid streams from respective ports are delivered at sufficient flow rates (and patterns) to create cavitation and/or other preferential effects to improve removal of clot. In certain examples, the cavitation effect is created by large pressure drops and deceleration at the focal point and/or intersection point of at least two fluid streams. The cavitation may provide a source of turbulent kinetic energy that can be used to mechanically fractionate and/or liquefy thrombi or other target tissue structures. When the fluid velocity is sufficiently high, the material accumulates impact energy, which can cause deformation and fragmentation. This also may modify the surface properties of the clot to allow the material to be penetrated to enable cavitation within the clot. Collision or interaction of the high-speed jets creates hydrodynamic cavitation whereby a pressure drop below the vapor pressure of the liquid creates bubbles which eventually collapse with great mechanical energy in the cavitation field, causing a kind of implosion in the clot material. Further, with multiple jets directed towards a focal point or sufficiently near respective streams, the closing speed of the fluid particles is significantly higher (up to double) that of a single jet stream. This also forces fluid and/or particles out from the space between the fluid jets at high speed. The speed of the fluid jets is sufficiently high to create a pressure drop below the vapor pressure such that the fluid vaporizes. When pressure rises again the bubble collapses, which causes the cavitation. It has been found that the power of the exemplary system and cavitation effect significantly exceeds conventional fluid jet(s) and mechanical tools like rotating screws. In some examples, the collapse of the bubbles may generate heat in or around the target tissue, which may further promote breaking up of the clot. In bench studies systems in accordance with various embodiments were able to remove certain clot material that simple aspiration or water jetting were not. In other studies, the exemplary systems were able to remove clot material in a fraction of the time of conventional systems.

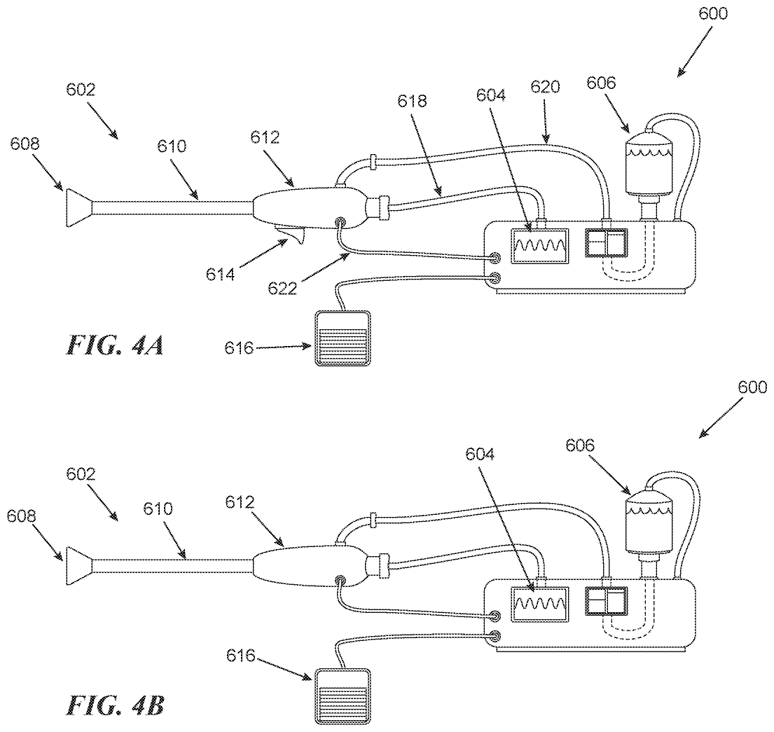

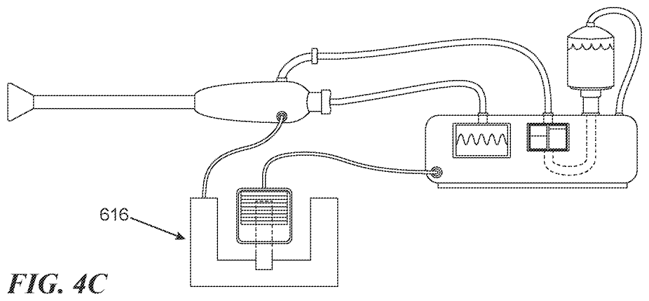

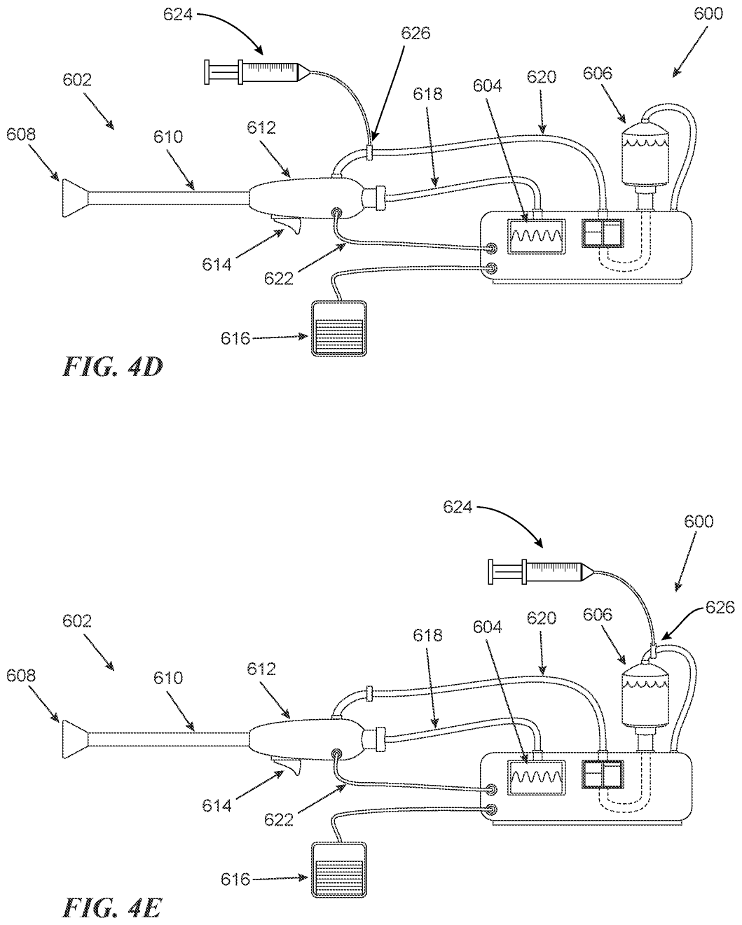

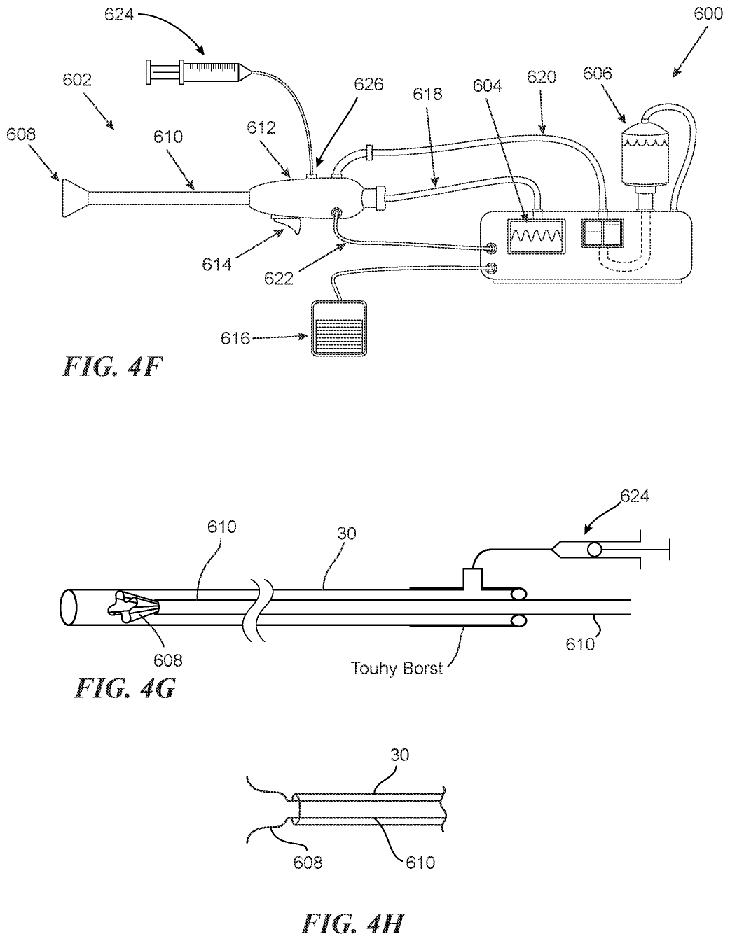

FIGS. 4A-4H illustrate various configurations of a thrombus removal system 600, including a thrombus removal device, 602, a vacuum source and cannister 604, a fluid source 606 and a pump 607. In some embodiments, the vacuum source and cannister and the fluid source are housed in a console unit that is detachably connected to the thrombus removal device. A fluid pump 607 can be housed in the console as shown, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 616, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

FIGS. 4D-4H illustrate embodiments of a thrombus removal system 600 that can additionally include a contrast injector 624 fluidly coupled to the fluid line 620 distal to the fluid source 606 (FIG. 4D), proximal to the fluid source 606 (FIG. 4E), or to a fluid line or auxiliary lumen within the thrombus removal device that is separate or distinct from the fluid lines and jets/ports. (FIG. 4F). In various embodiments, the contrast injector can be automatically controlled. The contrast injector can be configured to direct, apply, or inject a volume of a contrast agent into the subject's blood stream for the purpose of visualizing one or more clots in the subject and/or the relative position of a thrombectomy device to the one or more clots. Herein controlled can include any or any combination of pressure, rate, or volume. The contrast agent can be used, for example, to assist with positioning the thrombectomy device, and/or to provide information to a user regarding hemodynamic state, vascular anatomy, and or treatment progress/completion.

In some examples, the contrast agent can be immunologically tagged to specifically marker or be designed to be attracted to or accumulate on or within a target thrombus. For example, immunologically tagging a contrast agent to a blood clot may include attaching molecules that can specifically bind to components of the blood clot, thereby enhancing the visualization of the clot during medical imaging procedures. This technique can be used with medical imaging modalities such as computed tomography (CT), magnetic resonance imaging (MRI), or ultrasound, where the contrast agent helps highlight specific structures within the clot(s).

In some aspects, a molecule can be identified that is uniquely present on or near the blood clot. This molecule can be absent or minimally present in healthy tissues to ensure the specificity of the tagging process. Common targets include fibrin, a protein that forms the structural framework of blood clots, and other clot-specific proteins or receptors. The contrast agent can be designed to carry the immunological tag. The contrast agent could be a small molecule, nanoparticle, or even a specially designed biomolecule. The immunological tag, often an antibody or a smaller antibody fragment called a Fab fragment, can then be chemically attached to the contrast agent. This attachment can be achieved through various chemical coupling techniques, such as biotin-streptavidin interactions, covalent bonding, or other specific binding mechanisms. According to aspects of the present disclosure, the immunologically tagged contrast agent can administered to the subject with the contrast injector described above. Depending on the imaging modality, the tagged agent may be injected into the bloodstream (e.g., with the contrast injector), ingested, or introduced through other appropriate routes. As the contrast agent circulates through the vasculature, it is configured to bind to the blood clot or its vicinity due to the specific interaction between the tag and the target molecule. The tagged clot can be viewed under medical imaging (CT, MRI, ultrasound, etc.) as usual. The tagged contrast agent, being localized to the blood clot area, is configured to enhance the visibility of the clot on the resulting images. This increased contrast makes the blood clot stand out against the surrounding tissues, aiding in accurate diagnosis and assessment of the clot's size, shape, and location. In some aspects, the contrast may be directly injected into the clot. In some embodiments, the contrast agent might be delivered as part of a thrombolytic pretreatment procedure.

The contrast injector 624 can be configured to automatically inject or deliver selected volumes or boluses of any contrast agent into the thrombus removal system to assist with imaging of the thrombus removal device and/or a target thrombus. In some embodiments, while the volumes and timing of contrast to be delivered by the injector are selected by a user or pre-selected, the injector can be configured to automatically and/or continuously deliver contrast at the selected volumes and frequency. In the illustrated embodiment, the contrast injector can comprise a cradle assembly configured to receive one or more contrast injection syringe(s). The cradle assembly can include an automatic pusher or other mechanism configured to engage with the syringe to inject a contrast agent into the fluid line of the thrombus removal system. As shown, the contrast injector is fluidly coupled with the fluid stream/jetting components of the thrombus removal system. Therefore, contrast delivered to the thrombus removal system in these embodiments can then be delivered into a patient through the fluid/jet ports previously described.

A valve 626 can be, for example, controlled by a processor or controller of the system to facilitate injection of a contrast agent into the fluid stream. When the valve 626 is in a closed state, fluid can pass from the fluid source 606 through fluid line(s) 620 into the previously described fluid lumens of the thrombus removal device, and then through the jets or ports to facilitate cutting or macerating clots captured in the funnel of the device. However, when the valve 626 is in an opened state, contrast agent from the contrast injector can be allowed to enter a fluid lumen of the thrombectomy device (e.g., fluid line 620) for delivery or injection into the patient at the funnel location (e.g., through the jet ports).

In the embodiment of FIG. 4D, the delivery of fluid from the fluid source 606 can be paused while contrast is being injected into the system, allowing only the concentration of contrast agent from the contrast injector to be delivered into the patient. In other embodiments, a selected amount or volume of fluid from the fluid source can be blended or mixed with the contrast agent from the contrast injector for delivery to the target location. The amount of fluid mixed with the contrast agent can be controlled to provide the desired or appropriate concentration of contrast for imaging. In some embodiments, fluid from the fluid source can be selectively mixed or blended with contrast agent from the contrast injector to provide different concentrations of the contrast agent depending on the specific densities or types of clots to be imaged. For example, a higher concentration of contrast agent may provide better visualization of very firm or hard portions of clots (e.g., a leading edge of the clots), while lower concentrations of contrast agent may be sufficient to provide visualization of softer or more compliant portions of clots. In this manner, if the concentration of contrast agent that is injected is not providing adequate visualization, the concentration can be adjusted and another bolus of contrast can be injected to better visualize the clot.

In the embodiment of FIG. 4E, however, the contrast injector delivers contrast agent proximal to the fluid source 606, so any contrast agent injected into the system is blended or mixed with the fluid within the fluid source. The embodiment of FIG. 4E does not provide for the ability to deliver contrast to a target location with the jet ports independent from jetting or fluid delivery, but does still provide for the ability to produce a known concentration of contrast agent in the fluid source (e.g., with knowledge of the volume of fluid in the fluid source and the amount of contrast agent injected into the fluid line by the contrast injector).

While FIGS. 4D and 4E describe delivering contrast agent into the fluid line distal and proximal to the fluid source, respectively, it should be understood that in other embodiments the contrast agent can be injected directly into the fluid source.

Alternatively, in FIG. 4F, the contrast injector 624 can be fluidly coupled to a fluid line or auxiliary lumen within the thrombus removal device that is separate or distinct from the fluid lines and jets/ports. For example, FIGS. 1B-1K described above provide numerous embodiments in which the shaft of the thrombus removal device can include one or more fluid lumens fluidly coupled to the jet ports, and additionally provides some embodiments that include additional auxiliary lumens not used for jetting or fluid delivery. Therefore, the embodiment of FIG. 4F provides the ability to delivery fluid through the fluid lumens and jets to break up or macerate clots, and additionally provides separate lumens within the shaft for contrast agent delivery separate from the fluid delivery or jetting. As with the other embodiments, a valve 626 can be controlled to allow delivery of contrast agent from the contrast injector 624 into the device (e.g., into the auxiliary or contrast delivery lumen of the device). In this example, the auxiliary lumens can be fluidly coupled to contrast delivery ports or openings near a distal end of the device (e.g., near a distal end of the shaft or alternatively in the funnel itself).

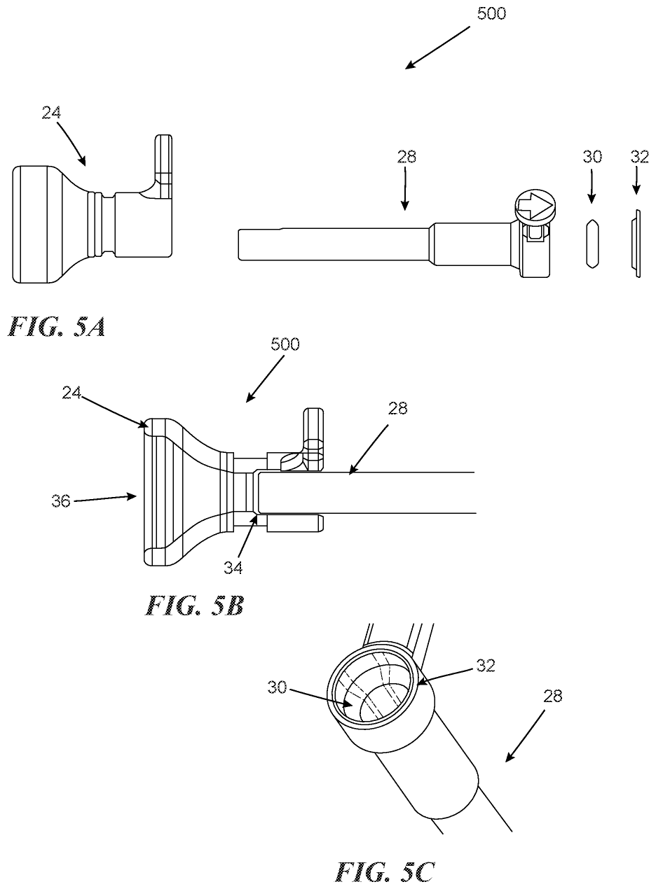

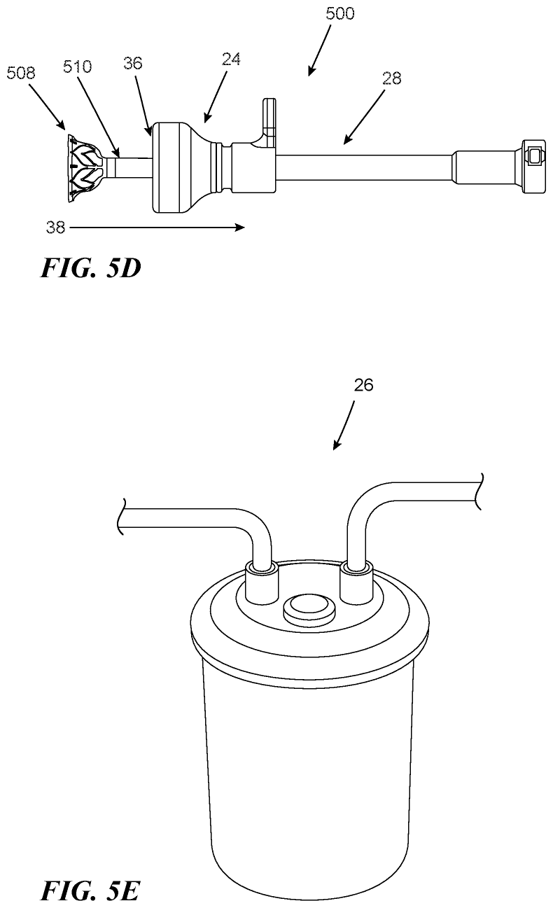

In another embodiment, as shown in FIGS. 4G-4H, instead of delivering the contrast agent into an auxiliary lumen of the thrombus removal device, the contrast agent can instead be delivered into the annular space between the thrombus removal device shaft and an introducer sheath or catheter used for delivery and navigation of the thrombus removal device to a target clot location. Additional details on the introducer sheath are described below, particularly in FIGS. 5A-5D, 6A-6E, and 7A-7C.

In FIG. 4G, a system assembly is shown including a funnel 608 and a flexible shaft 610 of a thrombus removal device inserted into a steerable introducer catheter 31. A hub assembly such as a Touhy Borst is shown which can provide access for a medical device into the steerable introducer catheter and include an injection port for fluidic connection to the contrast injector 624. In this embodiment, injection of contrast from the injector 624 into the hub assembly provides the contrast agent into the annular space between the introducer catheter 31 and the thrombus removal device (e.g., the shaft of the thrombus removal device). FIG. 4H shows the funnel 608 of the thrombus removal device axially disposed out of a distal end of the introducer catheter 31. In this example, contrast delivered by the injector 624 into the annular space can still be delivered into the patient, even when the funnel is in a deployed configuration. In some examples, the funnel can disperse the contrast agent as it's delivered past the funnel from the annular space. While the embodiment of FIGS. 4G-4H describes a thrombus removal device inserted into the introducer catheter 31, it should be understood that any elongate or catheter-based medical device can be inserted into the introducer catheter and used with the contrast injector 624. The contrast agent can be deployed in the annular space between the medical device and the introducer catheter as described above. The medical device can also include an expandable element (such as, but not limited to, a funnel) which can be compressed or collapsed within the introducer catheter. Alternatively, a dilator device can be inserted into the introducer catheter, as will be described below.

The contrast injector 624 of FIGS. 4D-4H can employ control algorithms or protocols to provide consistent or controlled injection of contrast agent near the distal end of the thrombus removal device. In some embodiments, the contrast injector can be configured to inject a pre-determined or pre-selected bolus or volume of contrast agent into the patient at the target thrombus location. For example, the contrast injector may be configured to a bolus of contrast agent (e.g., a 5 ml bolus or “shot” of contrast) at a pre-determined time interval (e.g., every 3-5 seconds). In some embodiments, the amount and frequency of the bolus of contrast agent delivered depends on the system state of the thrombus removal device. The amount and frequency of contrast agent delivery can therefore be tied to or triggered by the sensed state of the device or system during a clot removal procedure. For example, if the thrombus removal device is in a “clot hunting” state where no thrombus is engaged with the funnel, jetting is turned off or is minimal, and aspiration is turned off or is minimal, the contrast injector may be configured to deliver larger boluses of contrast agent with lower frequency (e.g., 10 ml boluses or “shots” of contrast every 5-10 seconds). Alternatively, if the system detects that a clot is engaged with the funnel and ramps up aspiration and jetting to clear the clot, the contrast injector may be configured to deliver smaller boluses of contrast agent with a higher frequency (e.g., 5 ml boluses or “shots” every 1-3 seconds). Once the system determines that the clot has been cleared, the contrast injector can again slow down the pace and volume of contrast agent delivery, or alternatively turn off contrast delivery altogether.

While the above embodiments describe a fluid connection between the contrast injector and the thrombus removal device, it should be understood that some embodiments can include two or more of the fluidic connections between the injector and the device. For example, a series of valves and fluid lines could be used to connect the contrast injector to two or more points along the fluidic path of the device. For example, the fluid lines could include connections to the system proximal to the fluid source, distal to the fluid source, and also to non-fluid lumen connections such as to auxiliary lumens or to the annular space between the introducer catheter and the shaft of the thrombus removal system. One or more electronic valves can then be controlled by the system to determine where the contrast agent enters the system. For example, a valve between the injector and the fluid lines distal to the fluid source could be opened to deliver a bolus of contrast directly into the fluid lines to be injected into the patient with the jet ports. Alternatively, a valve between the injector and an auxiliary lumen could be opened to deliver a bolus of contrast agent into the auxiliary lumen, to facilitate contrast delivery independent of jetting. Additionally, prior to treatment, a valve between the injector and the annular space between the introducer and shaft could be opened to allow for contrast injection through the dilator/introducer prior to deploying the thrombus removal device.

As is described above, aspiration occurs down the central lumen of the device and is provided by a vacuum pump in the console. The vacuum pump can include a container that collects any thrombus or debris removed from the patient.

FIG. 5A-5D illustrate one embodiment of a medical device loading tool 500, configured to load a medical device, such as a thrombectomy catheter, into an introducer sheath. As described herein, a thrombectomy catheter can include a funnel at a distal end of an elongate shaft. The medical device loading tool 500 can be configured to collapse the funnel of the thrombectomy device for insertion into the introducer sheath, and delivery into the subject.

FIG. 5A is an exploded view of a medical device loading tool 500. The loading tool can include a funnel introducer 24 and an introducer shuttle 28. The funnel introducer can comprise a distal opening 36 and a proximal opening configured to receive the introducer shuttle. An inner diameter of the funnel introducer proximal opening can be configured to receive an outer diameter of the introducer shuttle. Collectively, the funnel introducer 24 and the introducer shuttle 28 can be configured to load a thrombectomy device such as those discussed herein into an introducer sheath or catheter. The funnel introducer and introducer shuttle can be configured to compress a distal expandable portion (e.g., a funnel) of a thrombectomy catheter from an expanded/deployed configuration into a collapsed/delivery configuration. The medical device loading tool can further include an O-ring or seal 29 and a seal cap 32 configured to be mounted to a proximal end of the introducer shuttle. The medical device loading tool advantageously reduces the loading force required to sheath the funnel 20 within the introducer shuttle 28.

FIG. 5B shows the medical device loading tool 500 with a distal tip of the introducer shuttle 28 inserted into a proximal portion of the funnel introducer 24. The funnel introducer can include a distal opening 36 that is generally shaped and configured to receive a funnel of a medical device. In some embodiments, the shape of the distal opening can correspond to or be similar to a shape of the funnel. In some aspects, the shape of the distal opening can comprise a slightly narrowed or collapsed version of the shape of the funnel. The distal opening can be configured to reduce a loading force of the funnel into the introducer shuttle. In some embodiments, the proximal portion of the funnel introducer 24 can include a chamfered edge 34, which can provide a smooth lead-in from the funnel introducer into the introducer shuttle. When the medical device (such as a thrombectomy device) is inserted into the medical device loading tool and pulled proximally into the tool, the distal opening 36 of funnel introducer 24 first engages with a funnel or expandable element of the medical device to collapse or partially collapse the funnel or expandable element, and then continued proximal pulling of the medical device into the tool fully collapses or loads the funnel or expandable element into the introducer shuttle. When the funnel or expandable element is in the introducer shuttle, the funnel or expandable element can be considered to be in a collapsed or delivery configuration.

FIG. 5C is a proximal view of the introducer shuttle 28, showing the O-ring or seal 29 and seal cap 32. As shown, the proximal end can be filleted or atraumatic to avoid hard edges that may damage the medical device as it is pulled through the loading tool and introducer shuttle.

FIG. 5D shows a medical device loaded into the medical device loading tool 500. The medical device can comprise, for example, a thrombectomy device having an expandable element 508 such as an expandable funnel. As shown, a proximal end of the medical device catheter shaft 510 can be inserted into opening 36 of the funnel introducer 24 and extend through the proximal end of the introducer shuttle 28. In some embodiments, the medical device can further be primed before being loaded into the loading tool. To prime the device, the device can be submerged into a water or fluid bath and the jets or irrigation ports of the device can be activated to push water out through the jet ports/irrigation ports into the water bath. Next, the aspiration source can be turned on to pull water in through the aspiration lumen of the device. Referring to FIG. 5E, a waste container 26 is shown which can be fluidly coupled to the aspiration lumen of the device. In some embodiments, the user can monitor the waste container during the priming process. When air bubbles no longer present in the line leading to the waste container, or in the container itself, then the thrombus removal device is primed. In other embodiments, this process can be automated, such as by running the priming sequence for a set period of time, or alternatively, by mounting sensors (such as optical sensors) in the line leading to the waste container or in the container itself to monitor for the presence of air bubbles. When air is no longer detected by the sensor(s), the system can automatically turn off the priming sequence.

Referring back to FIG. 5D, the medical device can be pulled proximally into the medical device loading tool 500. First, the funnel introducer 24 collapses or partially collapses the funnel or expandable element 508 of the medical device. Next, the medical device is further pulled proximally (indicated by arrow 38) into the introducer shuttle 28, to place the funnel or expandable element 508 into a delivery or collapsed configuration within the introducer shuttle. When the funnel of the medical device is sheathed within the introducer shuttle, the funnel shuttle can be removed or torn away from the introducer shuttle. The primed and collapsed medical device can then be inserted into an introducer sheath with the introducer shuttle. In some aspects, the primed and collapsed medical device can be inserted into a Touhy Borst, such as in FIG. 4B, to load the medical device into the introducer sheath. Once the device is loaded in the sheath, the introducer shuttle can remain in place engaged with the Touhy Borst, or can be removed from the medical device (e.g., torn away). At this stage, the medical device (e.g., thrombus removal device) is primed and the funnel is sheathed and ready for insertion into a patient.

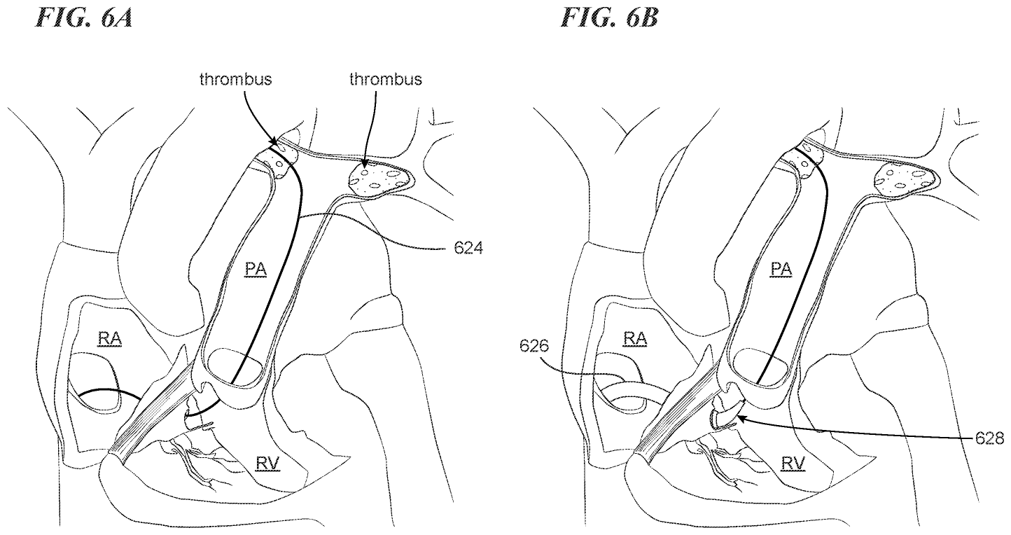

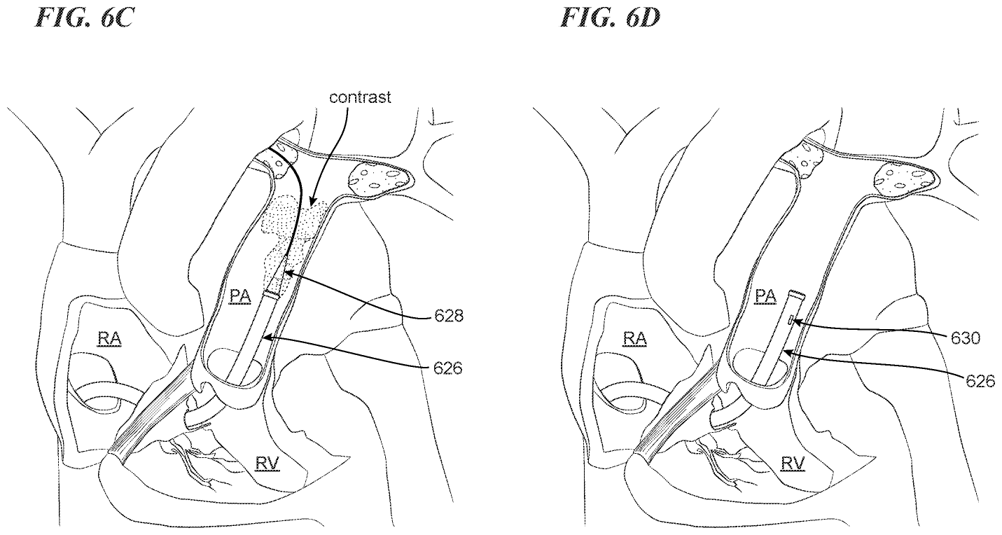

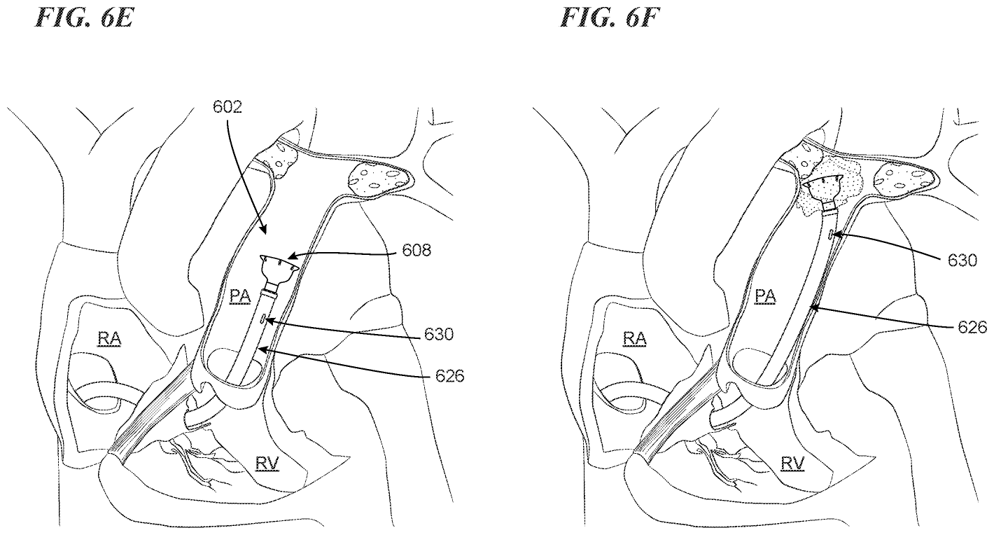

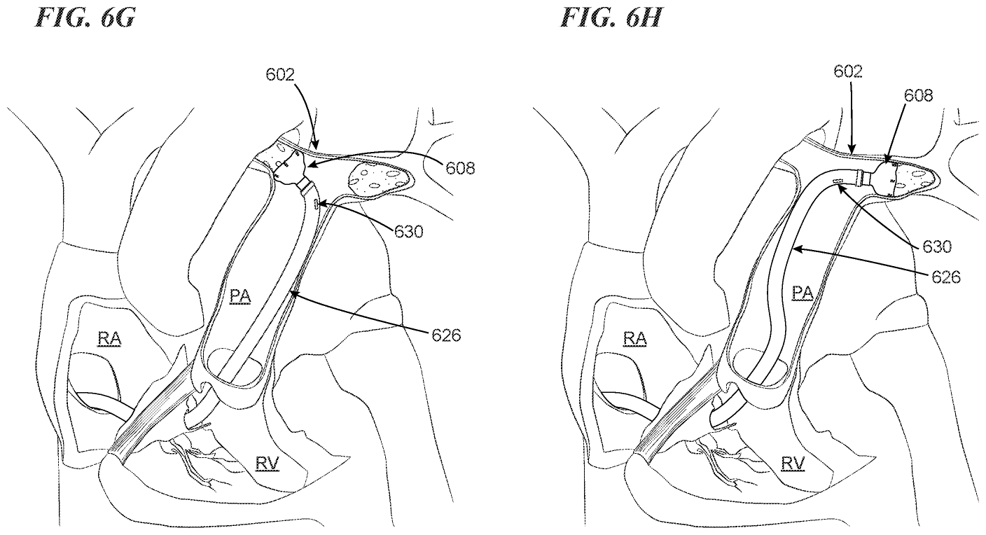

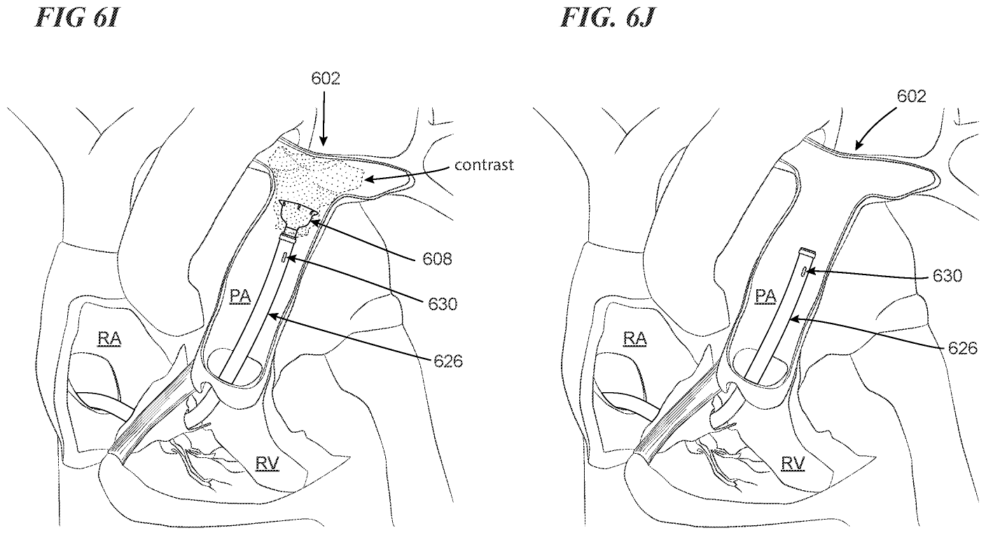

FIGS. 6A-6E illustrate a sequence of advancing a delivery catheter and dilator to a target thrombus location within a patient. In the illustrated example, one or more thrombi comprises one or more pulmonary embolism (PE) located in the pulmonary artery (PA) of a patient.

In FIG. 6A, a guidewire 624 can be inserted into the patient's vasculature and advanced towards the target thrombus location in the patient. For example, the guidewire may be inserted into a femoral vein of the patient, and routed into the pulmonary artery via the right atrium (RA) and the right ventricle (RV). In some examples, the guidewire 624 is passed through one or more thrombi in the pulmonary artery.

Next, referring to FIG. 6B, a dilator 628 and an introducer catheter 626 can be advanced into the patient over the guidewire. In FIG. 6B, the dilator 628 is shown in the RV prior to being advanced into the PA.

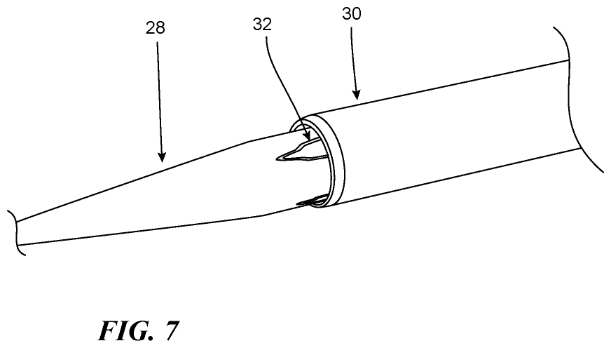

FIG. 6C shows the dilator 628 and introducer catheter 626 advanced further over the guidewire into the pulmonary circulatory system, such as the pulmonary artery (PA). When the dilator and introducer catheter are in the pulmonary artery, a contrast media or agent can be delivered into the pulmonary artery, as shown. The contrast media can be, for example, a “puff” or bolus of contrast media that can disperse within the pulmonary artery to provide imaging of any thrombi in the vicinity. In another embodiment, the contrast can be delivered continuously or in a “stream” such as to characterize blood flow as opposed to highlighting a clot. In one embodiment, the contrast can be injected directly by the introducer catheter 626 through one or more spaces between the dilator 628 and the introducer catheter. As described above, a contrast injector can be fluidly coupled or connected to the annular space between the thrombus removal device shaft and the introducer catheter or sheath. The contrast injector can be configured to automatically and/or continuously deliver selected volumes of contrast agent into this annular space and through the dilator. Referring to FIG. 7, in one implementation, the dilator 728 can include grooves 732, slits, or ports, or openings in fluid communication with a contrast source to facilitate contrast injection from the introducer catheter 726 when the dilator is in place. For example, contrast can be delivered through the introducer catheter and between the dilator and the introducer catheter.

At FIG. 6D, the dilator can be removed from the introducer catheter, leaving only the introducer catheter 626 positioned at a location proximal to the target thrombus or thrombi in the PA. In some embodiments, the introducer catheter can include on or more pressure sensors 630, positioned near a distal end of the introducer catheter. The pressure sensor(s) 630 can be configured to continuously or periodically obtain pressure measurements from within the vasculature, such as within the PA. In some embodiments, the pressure sensor(s) can provide a baseline pulmonary artery pressure prior to removing the thrombus. The pressure sensor can comprise, for example, a fiber optic pressure sensor. Other pressure sensor types are contemplated, including fluid column sensors. In some embodiments, the pressure sensor can be configured to continually monitor the PA pressure. During clot removal, the pressure sensor can provide useful information on the status of the procedure. For example, during removal, the pressure sensor can be used to monitor blood pressure in the vessel to characterize how effectively the thrombus removal system is removing clots. Typically, the baseline pressure when the clot is present will be elevated because blood cannot pass the clot. The pressure can continually be monitored during clot removal, and as the pressure drops it provides an indication to the user that the device has removed some or all of the clot.

In FIG. 6E, a thrombus removal device 602 can be advanced within the sheath and passed through a distal opening of the introducer catheter 626. Advancing the thrombus removal device out of the introducer catheter can allow the expandable funnel 608 of the thrombus removal device to deploy or expand within the PA. The thrombus removal device and the introducer catheter can be further advanced and steered towards the thrombi or clots, as shown in FIG. 6F. At this stage in the procedure, the user can optionally inject additional “puffs” or boluses of contrast agent into the vessel near the target thrombus location to visualize the clot and/or the introducer catheter. The contrast agent can be delivered in the annular space between the thrombus removal device catheter and the introducer catheter. With the funnel or expandable element 608 of the thrombus removal device deployed, the funnel can act to disperse the contrast agent near the thrombi. It should be noted that pressure measurements can be continuously or periodically taken within the PA during this and all other steps of the procedure.

In FIGS. 6G and 6H, the thrombus removal device can engage with the one or more clots in the PA and remove the clots. As described above, the clots can be removed with a combination of aspiration and jets/fluid streams delivered into the clot to break up or macerate the clot. Treatment progress can be monitored with on-demand “puffs” or boluses of contrast as needed. Additionally, pressure measurements can be obtained with pressure sensor 630 periodically or continuously, as described above.

In FIG. 6I, with the clots removed, the thrombus removal device and introducer catheter can be retracted proximally within the patient. Another optional “puff” or bolus of contrast agent can be delivered into the PA and former thrombus location to confirm that the targeted thrombi have been removed. Pressure measurements can be made with pressure sensor 630 as needed. In FIG. 6J, the thrombus removal device and funnel can be retracted into the introducer catheter, and the entire system including the introducer catheter can be removed from the patient.

In some aspects, the thrombus removal device can be configured to deliver boluses of contrast with pre-determined or user-selected contrast densities into a target thrombus location. The ability to adjust the density or concentration of delivered contrast agent can be used to improve or enhance visualization of clots, particularly complex clots with sections or portions of varying densities and stiffness. Injecting varying or differing contrast concentrations into a target thrombus location can provide clot localization and also provide information relating to a clot density profile. In one implementation, multiple boluses or “shots” of contrast agent with varying contrast concentrations can be delivered to a clot location. A gradient of images can then be formed from the variegated and sequential injection of varying contrast densities. For example, a first bolus (e.g., 5 ml) of contrast agent having a first contrast density can be injected to a target clot location. A first image of the clot can be captured, with the first image highlighting sections or portions of the clot that are well suited or tailored to absorbing or reacting with the first contrast density. Next, a second bolus (e.g., 5 ml) of contrast having a second contrast density can be injected to the target clot location. A second image of the clot may highlight or image different sections or portions of the clot compared to the first image. This can be repeated until the entire clot is imaged. In some examples, the various images of the clot at various contrast densities can be combined with image processing techniques to form a complete image of the clot based on the various contrast densities.

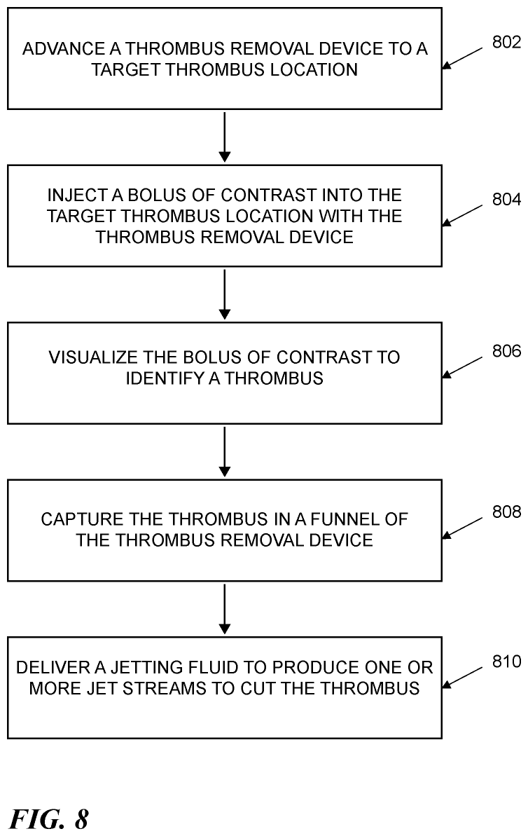

FIG. 8 is a flowchart describing a method of visualizing a thrombectomy procedure. Referring to step 802, the method can include advancing a thrombus removal device to a target thrombus location. In some examples, the thrombus removal device is delivered to the target location with an introducer catheter. In other embodiments, the introducer catheter gets the thrombus removal device in the vicinity of a target thrombus location, and then the thrombus removal device is advanced out of the introducer catheter.

At step 804, the method can include injecting a bolus of contrast into the target thrombus location with the thrombus removal device. In some examples, the contrast can be injected through one or more fluid lumens and fluid ports of the thrombus removal device. In other embodiments, the contrast can be injected or delivered through separate auxiliary or contrast lumens and associated ports of the thrombus removal device (e.g., jetting is separate from contrast delivery). Additionally, in another embodiment the contrast can be delivered from the annular space between an introducer catheter and the thrombus removal device (or alternatively between the introducer and a dilator).

At step 806, the method can include visualizing the bolus of contrast to identify a thrombus. Imaging can be any medical imaging configured to image a contrast agent, including but not limited to ultrasound imaging, CT, MRI, x-ray, or the like.

At step 808, the method can include capturing the thrombus in a funnel of the thrombus removal device. For example, aspiration can be activated in the thrombus removal device to pull the thrombus into a funnel of the thrombus removal device. In some embodiments, additional contrast can be delivered to confirm placement of the thrombus within the device.

At step 810, the method can include delivering a jetting fluid from a fluid source into the one or more fluid lumens to produce one or more jet streams from the one or more fluid ports to cut, macerate, or break up the thrombus. In some examples, aspiration can pull or remove the thrombus fragments from the patient.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method of visualizing a thrombectomy procedure, comprising:
advancing a thrombus removal device to a target thrombus location;
injecting a volume of contrast near the target thrombus location through one or more fluid lumens and fluid ports of the thrombus removal device;
visualizing the volume of contrast to identify a thrombus;
capturing the thrombus in a funnel of the thrombus removal device; and
delivering a jetting fluid from a fluid source into the one or more fluid lumens to produce one or more jet streams from the one or more fluid ports to cut the thrombus.

2. The method of claim 1, wherein injecting the volume of contrast further comprises injecting the volume of contrast with a contrast injector of the thrombus removal device.

3. The method of claim 1, wherein the volume of contrast enters the one or more fluid lumens distal to the fluid source.

4. The method of claim 1, wherein the volume of contrast enters the one or more fluid lumens proximal to the fluid source.

5. The method of claim 1, wherein the volume of contrast enters the one or more fluid lumens at the fluid source.

6. The method of claim 1, wherein the volume of contrast comprises less than 5 ml.

7. The method of claim 1, wherein the volume of contrast comprises less than 10 ml.

8. The method of claim 1, wherein the volume of contrast comprises less than 20 ml.

9. The method of claim 1, further comprising repeating the injecting and visualizing steps periodically.

10. The method of claim 9, further comprising repeating the injecting and visualizing steps every 1-3 seconds.

11. The method of claim 9, further comprising repeating the injecting and visualizing steps every 3-5 seconds.

12. The method of claim 1, further comprising adjusting a concentration of the contrast with the jetting fluid.

13. The method of claim 1, further comprising delivering two or more concentration densities of contrast into the target thrombus location to produce a gradient of images of the thrombus.

14. The method of claim 1, further comprising re-positioning the thrombus removal device adjacent to the thrombus based on the visualizing.

15. The method of claim 1, wherein the target thrombus location is located within a pulmonary artery.

16. A method of visualizing a thrombectomy procedure, comprising:
advancing a thrombus removal device to a target thrombus location;
injecting a volume of contrast near the target thrombus location through one or more contrast lumens and contrast ports of the thrombus removal device;
visualizing the volume of contrast to identify a thrombus;
capturing the thrombus in a funnel of the thrombus removal device; and
delivering a jetting fluid from a fluid source into one or more fluid lumens to produce one or more jet streams from one or more fluid ports to cut the thrombus.

17. The method of claim 16, wherein injecting the volume of contrast further comprises injecting the bolus of contrast with a contrast injector of the thrombus removal device.

18. The method of claim 16, wherein the volume of contrast comprises less than 5 ml.

19. The method of claim 16, wherein the volume of contrast comprises less than 10 ml.

20. The method of claim 16, wherein the volume of contrast comprises less than 20 ml.

21. The method of claim 16, further comprising repeating the injecting and visualizing steps periodically.

22. The method of claim 21, further comprising repeating the injecting and visualizing steps every 1-3 seconds.

23. The method of claim 21, further comprising repeating the injecting and visualizing steps every 3-5 seconds.

24. The method of claim 16, wherein the one or more contrast lumens and contrast ports are distinct from the one or more fluid lumens and fluid ports.

25. The method of claim 16, further comprising delivering two or more concentration densities of contrast into the target thrombus location to produce a gradient of images of the thrombus.

26. The method of claim 16, further comprising re-positioning the thrombus removal device adjacent to the thrombus based on the visualizing.

27. The method of claim 16, wherein the target thrombus location is located within a pulmonary artery.

28.-59. (canceled)
